(19) United States
(12) Patent Application Publication
(10) Pub. No.: US 2014/0066904 A1
(43) Pub. Date: Mar. 6, 2014

(54) Packaged Catheter Assembly

(71) Applicant: Tim Young, Moorabbin (AU)

(72) Inventor: Tim Young, Moorabbin (AU)

(73) Assignee: Sayco Pty. Ltd., Moorabbin (AU)

(21) Appl. No.: 14/012,703

(22) Filed: Aug. 28, 2013

(30) Foreign Application Priority Data
Aug. 29, 2012 (AU) 2012101310

(51) Int. Cl. A61M 25/00

(52) U.S. Cl. CPC A61M 25/002
USPC 604/544

(57) ABSTRACT

The present invention relates to a packaged catheter assembly comprising: a catheter assembly comprising a catheter shaft and a catheter sleeve mounted on the catheter shaft; and packaging for the catheter assembly, in which the catheter assembly is packaged, wherein the packaging is openable from one end thereof and wherein the catheter sleeve is mounted on a distal end of the catheter shaft, and wherein the distal end of the catheter shaft and the catheter sleeve are disposed at or towards the openable end of the packaging.

### CROSS-REFERENCE TO RELATED PATENT APPLICATIONS

This patent application claims priority to Australian Application No. 2012101310, filed Aug. 29, 2012, the entire teachings and disclosure of which are incorporated herein by reference thereto.

### FIELD OF INVENTION

The present invention relates to a packaged catheter assembly. In particular, the invention relates to a packaged catheter assembly that includes a catheter shaft and catheter sleeve, these components being arranged in the package to facilitate handling of the catheter assembly in use.

### BACKGROUND OF THE INVENTION

Urinary catheters are known and generally include a latex, polyurethane, or silicone tube that is inserted into a patient's bladder via the urethra. Catheterisation allows the patient's urine to drain freely from the bladder for collection. Urinary catheters may also be used to inject liquids used for treatment or diagnosis of bladder conditions. A clinician or nurse usually performs the catheterisation procedure, but self-catheterisation is also possible. If long term catheterisation is needed, catheters that facilitate self-catheterisation are most desirable to give patients a greater degree of independence.

Urinary catheters may be permanent (indwelling catheters), or may be intermittent and removed after each catheterisation. A Foley catheter (indwelling urinary catheter) is retained by means of a balloon at the tip of the catheter that is inflated with sterile water. The balloons typically come in two different sizes: 5 cm³ and 30 cm³ and are commonly made in silicone rubber or natural rubber. An intermittent catheter, or Robinson catheter, is a flexible catheter used for short term drainage of urine. Unlike the Foley catheter, it has no balloon on its tip and therefore cannot stay in place unaided. These can be non-coated or coated, for example they may be coated with a hydrophilic coating, and are generally packaged ready for use. Coudé catheters are designed with a curved tip that makes it easier to pass the catheter through the curvature of the prostatic urethra. Hematuria (or haematuria) catheters are a type of Foley catheter used for Post-TURP hemostasis. This is useful following endoscopic surgical procedures, or in the case of gross hematuria. There are both two-way and three-way hematuria catheters (i.e. double and triple lumen). External, Texas, urisheath, and condom catheters are generally used for incontinent males and carry a lower risk of infection than an indwelling catheter.

For some patients the insertion and removal of a catheter causes discomfort or pain, so a topical anesthetic is sometimes used. As mentioned above, in many cases catheterisation is performed as a sterile medical procedure by trained, qualified personnel, using equipment designed for this purpose. In the case of intermittent self-catheterisation, patients are trained to perform the procedure themselves. Intermittent self-catheterisation is performed by the patient four to six times a day, using a clean technique in most cases. Incorrect technique may cause trauma to the urethra or prostate (male), urinary tract infection, or a paraphimosis in the uncircumcised male. For patients with spinal cord lesions and neurogenic bladder dysfunction, intermittent catheterisation (IC) is a standard method for bladder emptying. The technique is safe and effective and results in improved kidney and upper urinary tract status, lessening of vesicoureteral reflux and amelioration of continence. In addition to the clinical benefits, patient quality of life is enhanced by the increased independence and security offered by self-catheterisation.

In order to maximise the potential for sterile insertion of catheters and thereby minimise the chances of urinary tract infection during self-catheterisation, a number of designs for catheter packaging have been proposed. The designs generally aim to remove the need for direct physical contact with the catheter during insertion. For example, in some instances the catheter is packaged within a bag from which it is fed by a user via indirect contact through the bag. In this example, the bag may contain a lubricant to assist with feeding of the catheter. In other examples, the catheter is removable from a package and is provided with a sleeve through which the catheter may be fed. In this example, a user pinches the sleeve to indirectly engage the catheter and facilitate insertion.

The options that are currently available are generally relatively easy to use when the user or person inserting the catheter has full function and control of their hands. However, in many instances users of catheters have reduced or limited hand and/or arm function. In such cases, catheters provided with packaging and sleeves as discussed above can be difficult to insert.

The subject matter claimed herein is not limited to embodiments that solve any disadvantages or that operate only in environments such as those described above. Rather, this background is only provided to illustrate one exemplary technology area where some embodiments described herein may be practice.

### SUMMARY OF THE INVENTION

The present invention relates to a packaged catheter assembly comprising:

a catheter assembly comprising a catheter shaft and a catheter sleeve mounted on the catheter shaft; and

packaging for the catheter assembly, in which the catheter assembly is packaged,

wherein the packaging is openable from one end thereof and wherein the catheter sleeve is mounted on a distal end of the catheter shaft, and wherein the distal end of the catheter shaft and the catheter sleeve are disposed at or towards the openable end of the packaging.

The form of the packaging is not particularly limited, provided it is openable from one end. Preferably, the packaging comprises an elongate sleeve within which the catheter assembly is packaged, the elongate sleeve comprising two opposed elongate sheets of packaging material joined along edges thereof. In one embodiment, the elongate sleeve comprises a pull tab at the openable end thereof, the pull tab facilitating opening of the elongate sleeve. For example, the pull tab may include an aperture adapted to receive a finger or thumb of a user to facilitate opening of the elongate sleeve. In a particular embodiment, sides of the opposed sheets are adapted to part from each other on the application of force by the user, thereby facilitating opening of the elongate sleeve.

The form of the catheter shaft is not particularly limited and may include any conventional or future catheter shaft. In any case, the catheter shaft includes at least one inlet at the distal end. Generally, the catheter shaft will include a plurality of inlets at its distal end.

The catheter sleeve may take any suitable form. For example, the catheter sleeve in its simplest form may comprise a tubular body portion and a head portion, the catheter sleeve extending circumferentially around the catheter shaft with the head portion being orientated towards the distal end of the catheter shaft. Generally, the catheter sleeve is formed from a compressible resilient material and can be compressed by a user to indirectly engage the catheter shaft without direct physical contact between the user and the catheter shaft. In addition, the catheter sleeve may comprise gripping surfaces on opposing sides thereof, the gripping surfaces being provided with texture for facilitating gripping of the catheter shaft with the catheter sleeve.

In certain embodiments it may be advantageous for a lubricant to be included. As such, the packaged catheter assembly may further comprise a lubricant within the packaging which lubricates the distal end of the catheter shaft.

The present invention consists of features and a combination of parts hereinafter fully described and illustrated in the accompanying drawings, it being understood that various changes in the details may be made without departing from the scope of the invention or sacrificing any of the advantages of the present invention.

### BRIEF DESCRIPTION OF ACCOMPANYING DRAWINGS

To further clarify various aspects of some embodiments of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the accompanying drawings in which:

FIG. 1 illustrates the packaged catheter assembly of an embodiment of the invention.

### DETAILED DESCRIPTION OF THE PREFERRED EMBODIMENTS

The present invention provides a packaged catheter assembly. In particular, the invention relates to a packaged catheter assembly that includes a catheter shaft and catheter sleeve, these components being arranged in the package to facilitate handling of the catheter assembly in use.

Hereinafter, this specification will describe the present invention according to the preferred embodiments. It is to be understood that limiting the description to the preferred embodiments of the invention is merely to facilitate discussion of the present invention and it is envisioned without departing from the scope of the appended claims.

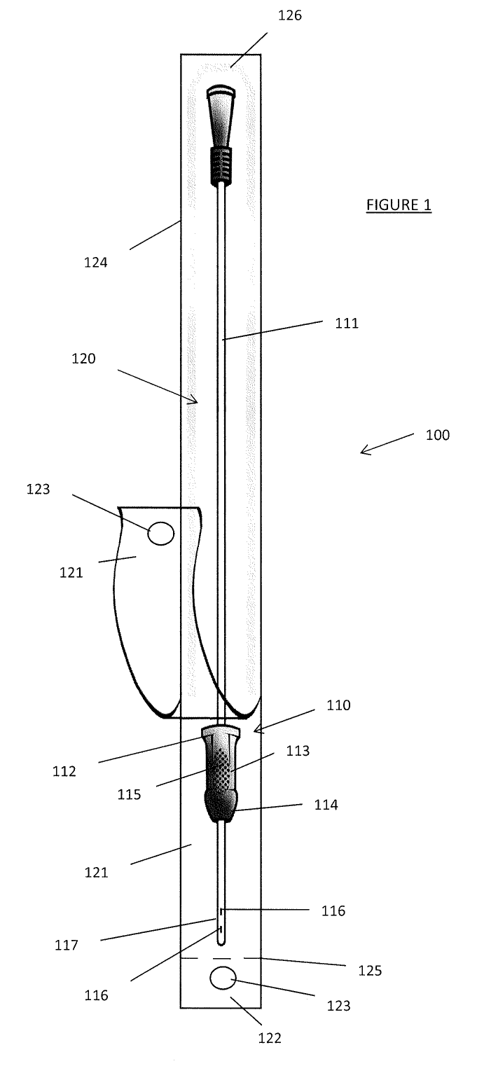

Referring to FIG. 1, a packaged catheter assembly 100 is provided. The packaged catheter assembly 100 includes a catheter assembly 110 and packaging 120. The form of the catheter assembly 110 is not particularly limited. In this embodiment, the catheter assembly includes a catheter shaft 111 and a catheter sleeve 112. The catheter sleeve 112 is tubular and extends circumferentially around the catheter shaft 111. In the illustrated embodiment, the catheter sleeve includes a body portion 113 and a head portion 114, although many other forms may be suitable. The body portion 113 is provided with opposed textured gripping surfaces 115 which facilitate indirect engagement of the catheter shaft 111 by a user of the catheter assembly 110.

The catheter shaft includes a plurality of inlets 116 at its distal end 117 which is to be inserted into the user of the catheter assembly 110. The catheter sleeve 112 is disposed on the distal end 117 of the catheter shaft 111, that is, the end of the catheter shaft 111 that is to be inserted into a user of the catheter assembly 110. Both the distal end 117 and the catheter sleeve 112 are disposed at one end of the packaged catheter assembly 100.

The packaging 120 includes two opposed elongate sheets 121, which are generally rectangular in shape, although other embodiments may be equally applicable. The opposed elongate sheets 121 are separable, and therefore the packaging 120 is openable at one end thereof 122. To assist opening of the packaging 120, the elongate sheets 121 are provided with apertures 123 which may receive a finger or thumb of a user to facilitate opening of the packaging 120. Advantageously, this may assist those users who have limited hand or arm movement.

The sides 124 of the elongate sheets 121 are joined, but can be easily parted by a user on the application of force, thereby enabling access to the catheter assembly 110. Generally, the sides 124 of the elongate sheets 121 will not be joined at the openable end 122. Rather, a join will be provided at a location 125 traversing the openable end 122 such that the apertures 123 can be gripped by a user to facilitate opening of the packaging 120. At the other end 126 of the packaging 120, the elongate sheets are permanently joined.

In use, a user of the catheter assembly 110 opens the packaging 120 by gripping the apertures 123 and tearing the elongate sheets 121 from each other along joined sides 124. This facilitates access to the catheter sleeve 112 and insertion of the distal end 117 of the catheter shaft 111 into the user. There is no need for manipulation of the catheter shaft 111 or catheter sleeve 112 as the distal end 117 of the catheter shaft 111 and the catheter sleeve 112 which indirectly engages the catheter shaft 111 are located at the openable end 112 of the packaging 120. This provides substantial advantages to the user of the catheter assembly 110, in particular those have limited hand or arm movement.

Unless the context requires otherwise or specifically stated to the contrary, integers, steps or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of steps, elements or integers. Thus, in the context of this specification, the term “comprising” is used in an inclusive sense and thus should be understood as meaning “including principally, but not necessarily solely”.

It will be appreciated that the foregoing description has been given by way of illustrative example of the invention and that all such modifications and variations thereto as would be apparent to persons of skill in the art are deemed to fall within the broad scope and ambit of the invention as herein set forth.

1. A packaged catheter assembly comprising:
a catheter assembly comprising a catheter shaft and a catheter sleeve mounted on said catheter shaft; and
packaging for said catheter assembly, in which said catheter assembly is packaged,
wherein said packaging is openable from one end thereof and wherein said catheter sleeve is mounted on a distal end of said catheter shaft, and wherein said distal end of said catheter shaft and said catheter sleeve are disposed at or towards said openable end of said packaging.

2. A packaged catheter assembly accordingly to claim 1, wherein said packaging comprises an elongate sleeve within which said catheter assembly is packaged, said elongate sleeve comprising two opposed elongate sheets of packaging material joined along edges thereof.

3. A packaged catheter assembly according to claim 2, wherein said elongate sleeve comprises a pull tab at said openable end thereof, said pull tab facilitating opening of said elongate sleeve.

4. A packaged catheter assembly according to claim 3, wherein said pull tab includes an aperture adapted to receive a finger or thumb of a user to facilitate opening of said elongate sleeve.

5. A packaged catheter assembly according claim 1, wherein said catheter shaft includes at least one inlet at said distal end.

6. A packaged catheter assembly according to claim 1, wherein said catheter sleeve comprises a tubular body portion and a head portion, said catheter sleeve extending circumferentially around said catheter shaft with said head portion being orientated towards said distal end of said catheter shaft.

7. A packaged catheter assembly according to claim 1, wherein said catheter sleeve is formed from a compressible resilient material and can be compressed by a user to indirectly engage said catheter shaft without physical contact between said user and said catheter shaft.

8. A packaged catheter assembly according to claim 7, wherein said catheter sleeve comprises gripping surfaces on opposing sides thereof, said gripping surfaces being provided with texture for facilitating gripping of said catheter shaft.

9. A packaged catheter assembly according to claim 1, further comprising a lubricant within said packaging which lubricates said distal end of said catheter shaft.
